(19) United States
(12) Patent Application Publication
(10) Pub. No.: US 2010/0280486 A1
(43) Pub. Date: Nov. 4, 2010

(54) SYSTEM AND METHOD FOR DELIVERING AND MONITORING MEDICATION

(75) Inventors: Mohammad M. Khair, Hoffman Estates, IL (US); Joanne M. Watt, Tower Lakes, IL (US); Steven R. Wehba, Carlsbad, CA (US)

Correspondence Address: BRIAN R. WOODWORTH, 275 N. FIELD DRIVE, DEPT. NLEG BLDG H-1, LAKE FOREST, IL, 60045-2579, US

(73) Assignee: HOSPIRA, INC., Lake Forest, IL (US)

(21) Appl. No.: 12/480,398

(22) Filed: Jun. 8, 2009

Related U.S. Application Data
(60) Provisional application No. 61/173,765, filed on Apr. 29, 2009.

(51) Int. Cl. A61M 5/168; G06F 17/00; G06Q 50/00; G06F 3/01; G06F 3/048

(52) U.S. Cl. 604/506; 235/375; 705/3; 715/702; 235/462.01; 715/764

(57) ABSTRACT

A method of delivering medication to a patient utilizing a medication delivery system is provided. A first medication to be delivered to the patient is supplied. The patient's identity is verified. The first medication is selected on a user interface to be delivered to the patient. The volume of the medication the patient will receive is entered. The first medication is injected into the patient through a flow sensor assembly. The flow rate and the volume of the first medication delivered to the patient are monitored by the flow sensor assembly while the injection occurs. A visual display provides information related to the injection. The method updates the patient's electronic medical administration record to capture the information regarding the injection of the first medication.

### CROSS REFERENCE TO RELATED APPLICATIONS

This application claims priority under 35 U.S.C. 119 of U.S. Ser. No. 61/173,765 filed Apr. 29, 2009.

### TECHNICAL FIELD

The present invention generally relates to a system and method for delivering and monitoring medication utilizing a system containing a flow sensor assembly,

### BACKGROUND

Modern medical devices, including medical pumps, are increasingly being controlled by microprocessor based systems to deliver fluids, solutions, medications, and drugs to patients. A typical control for a medical pump includes a user interface enabling a medical practitioner to enter the dosage of fluid to be delivered, the rate of fluid delivery, the duration, and the volume of a fluid to be infused into a patient. Typically, drug delivery is programmed to occur as a continuous infusion or as a single bolus dose.

It is common for a plurality of medications to be infused to a patient by using a multi-channel infusion pump or using a plurality of single channel infusion pumps where a different fluid is administered from each channel. Another method of delivering multiple medications to a patient is to deliver a first medication using an infusion pump, and additional medications through single bolus doses.

When delivering medications through single bolus doses it is important to verify that correct medications are being delivered to the patient as well to verify that the correct amount of medication is being delivered to the patient. Typically a caregiver simply manually notes on the patient's paper chart the amount of medication delivered via a bolus dose, and that information may later be entered into a patient's record electronically. Thus, human error may lead to an accidental overdose or underdose of a medication, while a caregiver believes that a proper dose was delivered. In addition to an error in medication dosing, it is also possible that human error may result in the failure to record the medication delivered during a single bolus dose. Thus, it is possible that a patient's medical records may not reflect every medication that patient has been given. A sensor within the IV line capable of measuring a wide range of fluids and flow rates would be helpful in documenting the flow rate and volume of every medication the patient is given through that line. Further, it is desirable to provide a robust flow rate sensing methodology that is low cost and in particular introduces low incremental cost to the disposable medication delivery tubing set. Further, it is desirable to provide a flow rate sensing methodology that is capable of accurately sensing the flow rate of fluids that have a range of physical properties, including fluid viscosity, which may not be known precisely. Additionally it is desirable to automatically record the medication that was delivered to the patient, as well as the dose of the medication, and the time at which the medication was delivered to the patient. Therefore, a need exists for a method that utilizes a fluid flow sensor system for monitoring medication delivery.

### SUMMARY

According to one aspect of the invention, a method of delivering medication to a patient utilizing a medication delivery system is provided. A first medication to be delivered to the patient is supplied. The patient's identity is verified. The first medication is selected on a user interface to be delivered to the patient. The volume of the medication the patient will receive is entered. The first medication is injected into the patient through a flow sensor assembly. The flow rate and the volume of the first medication delivered to the patient are monitored by the flow sensor assembly while the injection occurs. A visual display provides information related to the injection. The method updates the patient's electronic medical administration record to capture the information regarding the injection of the first medication.

According to another aspect of the invention, a method of delivering medication to a patient utilizing a medication delivery system is provided. A first medication to be delivered to the patient is supplied. The patient's identity is verified. A bar code on the first medication is scanned. The identity of the first medication is displayed based on scan of the bar code. The method confirms that the patient has been prescribed the first medication. The volume of the medication the patient will receive is entered. The first medication is injected into the patient through a flow sensor assembly. The flow rate and the volume of the first medication delivered to the patient are monitored by the flow sensor assembly while the injection occurs. A visual display provides information related to the injection. The method updates the patient's electronic medical administration record to capture the information regarding the injection of the first medication.

### BRIEF DESCRIPTION OF THE DRAWINGS

FIG. 1 is a pictorial view that illustrates a patient connected to IV line having a differential pressure based flow sensor assembly according to one embodiment;

FIG. 2 shows a closer, more detailed pictorial view of the differential pressure based flow sensor assembly of the embodiment of FIG. 1;

FIG. 3 is an isometric view of a differential pressure based flow sensor assembly of the embodiment of FIG. 1;

FIG. 4 is a cross-sectional view taken along line 4-4 of FIG. 3;

FIGS. 5a-5e illustrate cross-sections of flow restricting elements within differential pressure based flow sensor assemblies according to various embodiments;

FIG. 6 is a pictorial view illustrating delivery of medication to a patient via an IV push or bolus through an IV line having the differential pressure based flow sensor assembly of FIG. 1;

FIG. 7 schematically illustrates a method of delivering medication using a system having a differential pressure based flow sensor assembly according to one basic process;

FIG. 7a schematically illustrates a method of delivering medication using a system with a differential pressure based flow sensor assembly, according to a more elaborate process than FIG. 7;

FIGS. 8a-8b schematically illustrate a method of delivering medication using a system having a differential pressure based flow sensor assembly according to another process;

FIGS. 9-14 pictorially depict a method of a caregiver delivering medication to a patient using a system having a flow sensor assembly;

FIG. 15 is a pictorial view illustrating an alternate method of providing information to a system for delivering medication to a patient; and

FIGS. 16-28 pictorially depict screens that may be displayed on an infusion pump while mediation is about to be delivered or is being delivered to a patient.

### DETAILED DESCRIPTION

While this invention is susceptible of embodiments in many different forms, there is shown in the drawings and will herein be described an example of the invention. The present disclosure is to be considered as an example of the principles of the invention. It is not intended to limit the broad aspect of the invention to the examples illustrated.

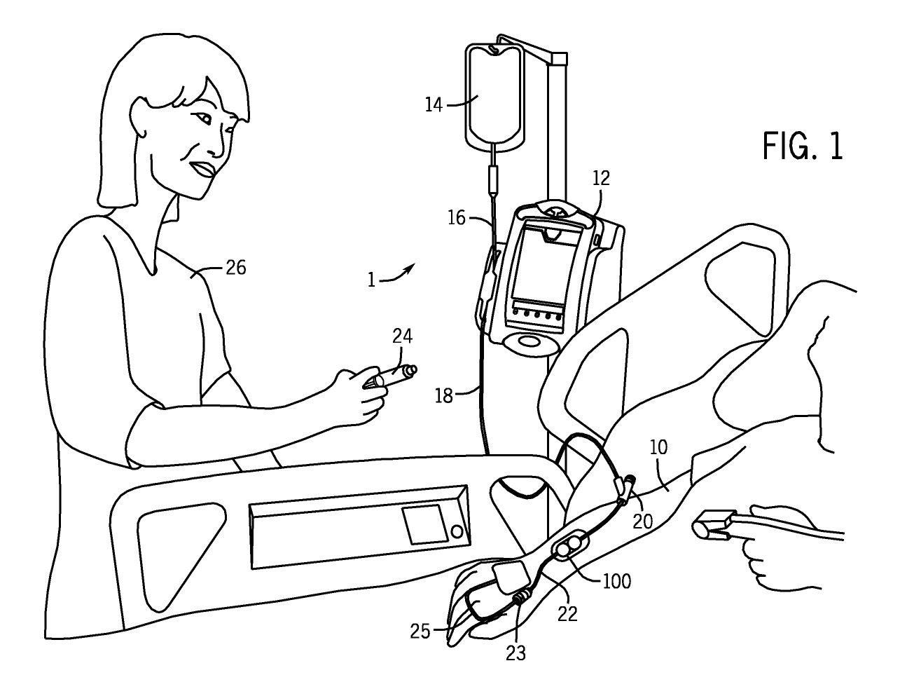

FIG. 1 is a pictorial representation of a patient 10 connected to a medication delivery system 1 and receiving a first medication via an infusion pump 12 from a medication reservoir 14. A first fluid line segment 16 delivers the first medication from the reservoir 14 to the infusion pump 12. The second fluid line segment 18 delivers the medication from the infusion pump 12 to a differential pressure based flow sensor assembly 100. A third fluid line segment 22 delivers the medication from the differential pressure based flow sensor 100 to the patient 10. While three fluid lines segments are described in connection with FIG. 1, it is contemplated that the number of fluid lines or line segments used in connection with the present invention may vary, and may be more or less than three fluid lines. The third fluid line segment 22 is typically connected to the patient 10 through a connector valve 23 and a patient access device such as a catheter 25.

The second fluid line segment 18 has a connection 20 adapted to receive a second medication from a second source. The connection illustrated in FIG. 1 is typically referred to as a Y-Site, although it is contemplated that other connection types and configurations may be used in connection with the present invention.

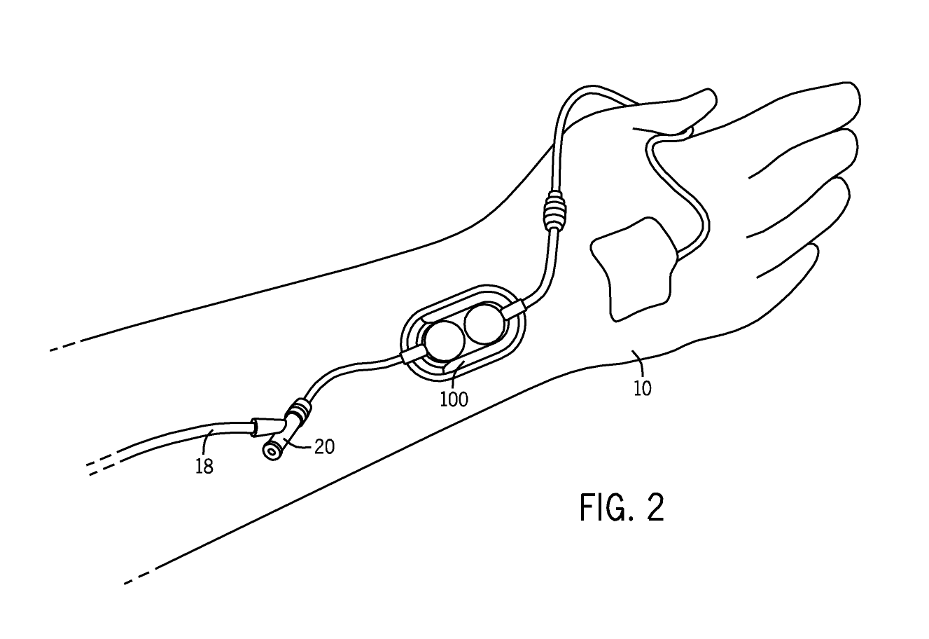

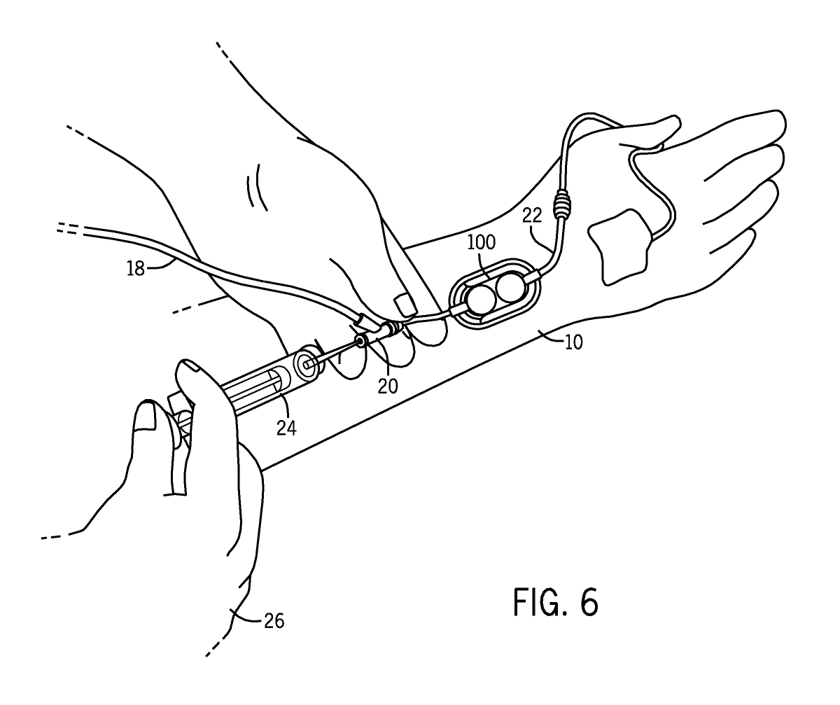

The connection 20, shown in additional detail in FIG. 2, may receive a second medication from a syringe 24 in the form of a manual IV push or bolus by a caregiver 26 (see FIG. 6). It is further contemplated that the second medication may be provided in another fashion, such as from a second medication reservoir or other known medication delivery source. The medication delivery system 1 further has a flow sensor assembly 100. In the illustrated embodiment, the flow sensor assembly 100 is a differential pressure based flow sensor assembly located downstream of the connector 20 and is secured on the patient 10. Thus, the flow sensor assembly is adapted to have one or more or medications, for example a first and a second medication, pass through the sensor assembly 100. However, the sensor assembly 100 could also be disposed in any number of locations including but not limited to upstream of the fluid junction between the first and second medication, connected between the second source and the connector 20, or integrally formed on or within one of the branches of the connector 20. The flow sensor assembly 100 need not be secured to the patient 10 directly.

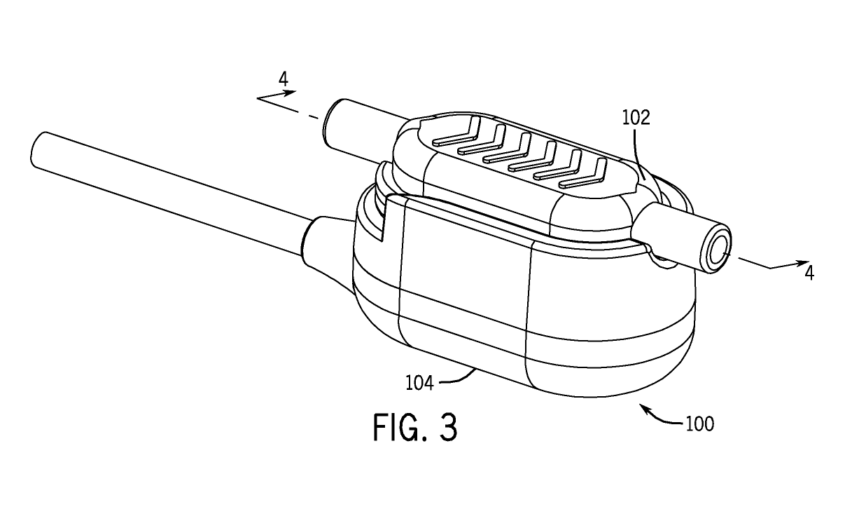

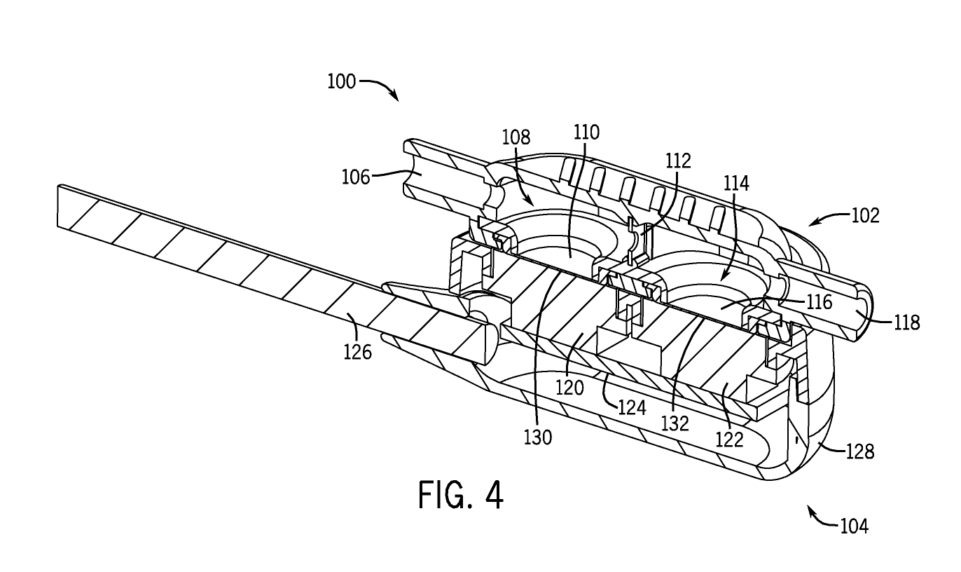

Turning next to FIG. 3 and FIG. 4, the differential pressure based flow sensor assembly 100 is shown in additional detail. The differential pressure based flow sensor assembly 100 has a disposable portion 102 and a reusable portion 104. As used herein reusable is defined as a component that is capable of being safely reused. For example, the same reusable portion 104 can be used multiple times on the same patient with the disposable portion 102 being changed at least every 72 hours or so. The same reusable portion 104 can be used hundreds or even thousands of times on different patients, subject to the cleaning policies recommended by the manufacturer or the healthcare institution, by installing a new disposable portion 102. This is possible since the reusable portion 104 is designed to prevent fluid ingress. As may best be seen in FIG. 4, the disposable portion 102 has a fluid inlet 106 an upstream fluid chamber 108, an upstream fluid pressure membrane 110, a flow restricting element 112, a downstream fluid chamber 114, a downstream fluid pressure membrane 116, and a fluid outlet 118. The membranes 110 and 116 are fluid impermeable. Although full membranes are shown, it is contemplated that other types of seals, including but not limited to one or more gaskets and O-rings, would suffice to keep fluid out of the housing of the reusable portion. Any exposed areas could be swabbed with a cleaning solution, if necessary.

As shown in FIG. 4, medication enters the disposable portion 102 through the fluid inlet 106. The medication flows into the upstream fluid chamber 108 from the fluid inlet 106. Next, the medication flows through the flow restricting element 112 and into the downstream fluid chamber 114. The flow of the medication through the flow restricting element 112 results in a drop in fluid pressure as the fluid flows from the upstream fluid chamber 108 to the downstream fluid chamber 114 through the flow restricting element 112. Thus, during forward fluid flow under normal conditions, the fluid pressure within the upstream fluid chamber 108 is generally greater the fluid pressure within the downstream fluid chamber 114. The fluid pressure within the upstream fluid chamber 108 presses against the upstream fluid pressure membrane 110. Similarly, the fluid pressure within the downstream fluid chamber 114 presses against the downstream fluid pressure membrane 116.

It is contemplated that a variety of materials may be utilized for the manufacture of the disposable portion 102. The disposable portion 102 may comprise a thermoplastic. It is contemplated that the flow restricting element 112 may be made of the same thermoplastic as the rest of the disposable portion 102, or may be a different material than the disposable portion 102. Non-limiting examples of the material that may be utilized to form the flow restricting element 112 include silicon, glass, and medical grade thermoplastics and elastomerics. The fluid pressure membranes 110, 116 may comprise a variety of polymeric or elastomeric materials, such as TPE, or silicone.

It is additionally contemplated that the flow restricting element 112 may be formed integrally with the rest of the disposable portion 10, or the flow restricting element 112 may be a separate component placed within the disposable portion 102.

As may also be seen in FIG. 4, the reusable portion 104 of the differential pressure based flow rate sensor assembly 100 has an upstream pressure sensor 120, a downstream pressure sensor 122, a circuit board 124, and an electrical connection 126, all contained within a housing 128. The upstream pressure sensor 120 is adapted to interact with the upstream fluid pressure membrane 110 to generate a reading of fluid pressure within the upstream fluid chamber 108. Similarly, the downstream pressure sensor 122 is adapted to interact with the downstream fluid pressure membrane 116 to generate a reading of fluid pressure within the downstream fluid chamber 114. The circuit board 124 receives output from both the upstream pressure sensor 120 and the downstream pressure sensor 122. A processor (not shown) on the circuit board 124 may calculate a pressure difference between the upstream fluid chamber 108 and the downstream fluid chamber 114, or the circuit board 126 may generate an output signal that is transmitted to another remote device with a processor, such as the infusion pump 12, that calculates the pressure difference between the upstream chamber 108 and the downstream chamber 114. Output of the circuit board 124 passes through electrical connection 126 to the infusion pump 12 (FIG. 1).

Although a wired electrical connection 126 is shown in FIG. 4, the system may optionally comprise wireless electrical connection and communication with the infusion pump 12 or other system components. It is additionally contemplated that according to some alternative embodiments, the reusable portion 104 may further contain additional electronics, such as, batteries, one or more memories, amplifiers, signal conditioning components, analog-to-digital converters, power converters, LED indicators, a display, sound generating components, a wireless communication engine, inductive coils for receiving power from the infusion pump 12 or another source, and active or passive radio frequency identification devices (RFID). It will be appreciated that the calculations and processing described herein can take place on the circuit board 124, in the infusion pump 12, in a remote processor (not shown), or be concentrated in only one of the system components, or distributed among one or more of the system components as needed or desired.

The components of the reusable portion 104 are contained within the housing 128. The housing 128 may be manufactured from a polymeric material such as polycarbonate, polyethylene, polyurethane, polypropylene, acrylic, or other known materials. It is further contemplated that an upstream reusable portion membrane 130 may separate the upstream fluid pressure membrane 110 from the upstream fluid pressure sensor 120. Likewise, a downstream reusable portion membrane 132 may separate the downstream fluid pressure membrane 116 from the downstream fluid pressure sensor 122.

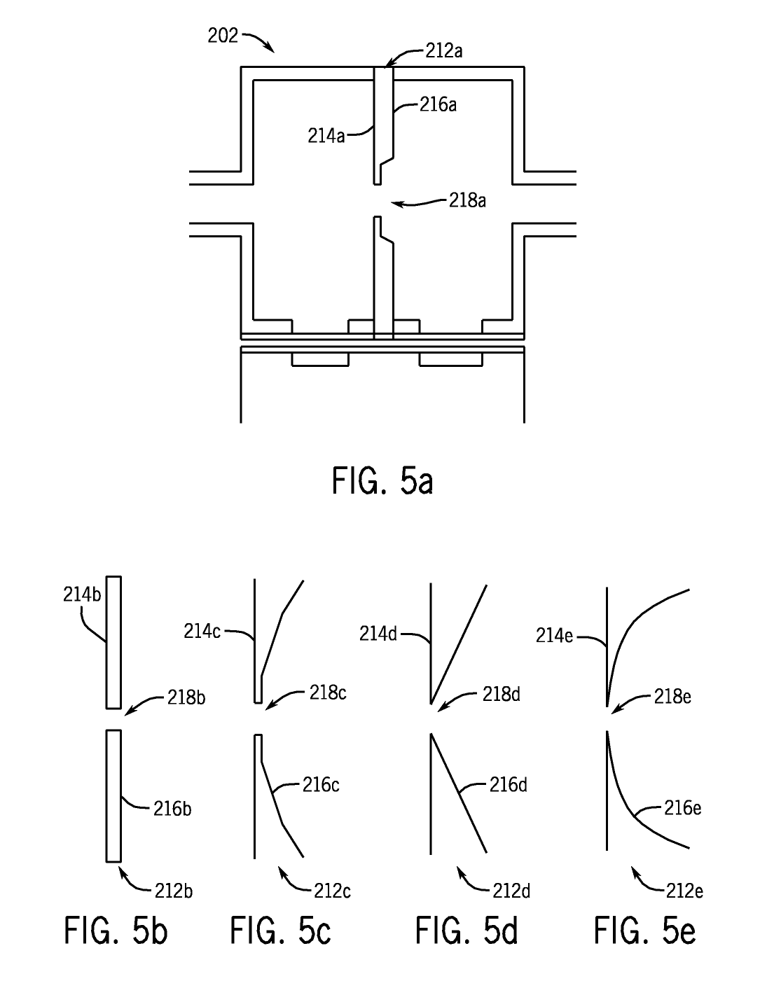

Referring next to FIG. 5a, a cross-section of a disposable portion 202 is schematically illustrated with a flow restricting element 212a to show one profile of the flow restricting element 212a. The flow restricting element 212a may be identical to the flow restricting element 112, but may also vary. The flow restricting element 212a is in the form of an orifice. An orifice may be a beneficial flow restricting element, as orifice performance varies less between fluids of different viscosities than other flow restricting elements, such as capillary channels. That is to say, the measured pressure differential across an orifice for a given flow rate will be largely independent of the viscosity of the active solution, where the pressure difference measured across alternate restrictions such as capillaries will demonstrate a strong dependence upon fluid viscosity. The flow restricting element 212a has a front face 214a located on an upstream side of the flow restricting element 212a, and a rear face 216a on the downstream side of the flow restricting element 212a. An opening 218a is formed through the flow restricting element 212a to allow fluid to flow through the flow restricting element 212a.

The opening 218a may have a variety of cross-sectional shapes, but a circular opening is commonly used. In order to help reduce the effect of fluid viscosity on the flow of the fluid through the opening 218a of the flow restricting element 212a, the opening 218a may have a ratio of a perimeter of the opening 218a to the length the fluid travels though the opening 218a of from about 100:1 to about 2000:1. That is, the perimeter of the opening is sufficiently larger than the length of fluid flow though the opening 218a, such that the pressure drop through the opening 218a is less dependent on the fluid, and more dependent on the geometry of the opening 218a. An opening 218a having a perimeter to flow length ratio of about 1000:1 has been found to be effective. For example, a 430 micron diameter circular orifice with a length in the flow dimension of 12 microns will accommodate flow rates in the hundreds to thousands of ml/hr. A smaller diameter orifice would be needed for smaller flow rates and applications.

The thickness of the opening 218a of the flow restricting element may vary from about 5 microns to about 25 microns. An opening 218a having a thickness of about 12 microns has been found to be effective. In order to demonstrate the desired flow characteristics, it is important to provide a flow orifice or opening in a solid geometry. The ratio of the inlet height to the effective hydraulic diameter of the orifice should be rather large, such as at least 10:4 or about 5:1. However, a constant-thickness membrane, of thickness equal to the length of the desired orifice, may become mechanically weak if the overall area of the membrane is large. Once the orifice opening is established, the membrane material in which the orifice resides can be thicker as one moves away from the orifice perimeter. As a result, the orifice itself can provide the desired restrictive fluid path length, while the membrane in which the orifice resides is thicker than the length of the orifice at a location away from the orifice. Thus, it is contemplated that various other geometries may also be used to form a flow restricting element.

As shown in FIG. 5a, the flow restricting element 212a transitions from a thicker cross sectional shape to a thinner cross sectional shape near the opening 218a. Creating such geometry for the flow restricting element 212a allows for various low cost manufacturing approaches for the flow restricting element 212a. Creating such geometry has a limited effect on performance of the flow restricting element 212a, as such geometry does not introduce a significant pressure difference for fluids having different viscosities, but having the same fluid flow rate. Thus, the thinness of the flow restricting element 212a near the opening 218a limits the effect of fluid viscosity on pressure drop through the opening 218a, while thicker material away from the opening 218a increases the overall strength of the flow restricting element 212a.

FIGS. 5b-5e illustrate alternative flow restricting elements 212b-212e that function similarly to flow restricting element 212a. Flow restricting element 212b maintains a constant thickness, while flow restricting elements 212c-212e are thinner near the openings 218c-218e. The geometry of the rear face 216a-216e is designed to have minimal effect on flow characteristics through openings 218a-218e. This is because flow through the opening 218a-218e typically features well-defined fluid velocity profiles with minimal fluid/wall dynamic interaction on the orifice backside, as long as the rear face 216a-216e geometry is sloped away from the orifice appropriately, and therefore minimizes viscosity induced pressure losses. Some of these orifice geometries lend themselves to manufacturing advantages. For example, orifice 218a can be formed efficiently via silicon processing techniques such as etching, lithography, masking and other MEMS operations. Orifice 218b can be formed efficiently by laser machining thin flat stock material. Orifices 218c and 218d could be formed easily with photo-imaging glass processing techniques. Orifices 218c, 218d, and 218e could be formed using molding or embossing techniques. Further combinations of techniques could be utilized within the scope of the invention.

While many embodiments have been described in connection with an upstream pressure sensor, a flow restricting element, and a downstream pressure sensor within a common assembly, it is further contemplated according to a further alternative embodiment, that these components may be separate standalone components within a fluid flow system. The methods and processes of measuring fluid flow rates and the volume of fluid flow are generally identical to those previously described according to this alternative embodiment. Thus, by monitoring the difference in pressure between a standalone upstream pressure sensor and a standalone downstream pressure sensor generated by fluid flowing through a standalone flow restricting element, the fluid flow rate may be calculated.

Turning next to FIG. 6, an IV push or bolus is shown being delivered to the patient 10. The caregiver 26 connects the syringe 24 to the second fluid line 18 via the connection 20. The caregiver 26 then delivers the mediation within the syringe 24 to the patient through the connection 20. The medication passes through the differential pressure based fluid flow sensor 100 and the third fluid line 22 to the patient 10. The differential pressure based fluid sensor assembly 100 monitors the flow rate of the medication through the sensor assembly 100. By monitoring the flow rate through the sensor assembly 100, the volume of medication delivered to the patient 10 may be calculated.

The flow rate of the fluid through the pressure sensor assembly 100 may be calculated by the following equation:

$$Q = A {C}_{D} \sqrt{\frac{2 Δ P}{ρ}} ,$$

where Q is the volumetric flow rate, ΔP is the pressure differential between an upstream pressure sensor and a downstream pressure sensor, ρ is the fluid mass density, C_{D} is an opening discharge coefficient, and A is the area of the opening. The use of an orifice for the opening has been empirically shown to minimize the dependence of the induced pressure differential on fluid viscosity, and the discharge coefficient remains essentially constant, thus making the flow rate a function of pressure, density, and area.

Once the flow rate Q has been calculated, the volume of the flow may be determined by integrating the flow rate over a period of time using the following equation: V=∫Qdt. Using this equation, both forward and backward flow thorough the sensor assembly 100 may be calculated. A negative flow rate would indicate that the pressure at the downstream sensor 122 is higher than the pressure at the upstream sensor 120, and thus fluid is flowing backwards through the sensor assembly 100, away from the patient 10.

In order to provide a more accurate ΔP, a pressure tare, or calibration of the sensors, may be performed, preferably in a zero flow condition. A pressure tare subtracts the average pressure of both the upstream pressure sensor 120 and the downstream pressure sensor 122 from the readings of the respective upstream and downstream pressure sensors 120, 122 during fluid delivery. Utilizing such a pressure tare reduces the occurrence of signal drifts from pressure supply drifts, amplification, temperature variance, or residual pressures from any priming steps prior to delivering and recording a bolus dose.

Reverse flow of fluid through the sensor can be also measured with ΔP being negative. In this case, the flow is computed by taking the absolute value of ΔP and moving the negative sign outside the square root,

$$Q = - A {C}_{D} \sqrt{\frac{2 \vert Δ P \vert}{ρ}} .$$

Negative flow rates are important to aggregate in the computation of true net forward volume delivery from the syringe, as they may impact the accuracy of total net volume delivered from the syringe. Additionally, an occlusion condition (i.e., the catheter 25 or the patient's vein being closed or occluded) can be detected using a back draw of the syringe prior to forward fluid delivery, a typical clinical practice. Under normal conditions, reverse flow of the fluid can be directly measured and aggregated into the net forward volume delivery. However, under occlusion scenarios, the occluded reverse flow can be quickly detected by the sensor using threshold negative limits of the downstream and upstream sensors drawing a negative vacuum pressure.

The outputs of the upstream pressure sensor 120 and the downstream pressure sensor 122 may further be monitored for detection of motion artifacts to distinguish such artifacts from true flow patterns. To detect motion artifacts, a ratio of the upstream pressure sensor 120 output to the downstream pressure sensor 122 output is monitored. If, for example, the ratio is less than a predetermined threshold, such as 3:1, it is likely that any changes in pressure indicated by the upstream pressure sensor 120 and the downstream pressure sensor 122 are the results of motion artifacts within the sensor assembly 100, not forward fluid flow. Thus, flow is only indicated when the ratio of the pressures indicated by the upstream pressure sensor 120 and the downstream pressure sensor 122 is greater than a threshold amount. This is because once flow is initiated, the flow restricting element 112 causes the pressure at the upstream pressure sensor 120 to be significantly higher than the pressure at the downstream pressure sensor 122. Alternatively, reverse fluid flow is similarly distinguished from motion artifacts, if the ratio of the downstream pressure sensor to the upstream pressure sensor is more than a limit threshold, such as 3:1, and otherwise the signal is considered motion artifacts. Pressure values obtained due to motion artifacts may be excluded from the flow rates and aggregate volume computation. Motion artifacts events are also distinguished from events indicating the true onset of flow, which is used to gate or determine the start of bolus delivery via the syringe 24.

Algorithms also are contemplated to detect the start and end of a single bolus dose. Such an algorithm may rely on a first derivative and a short term mean value of the flow rate. If the mean value of the flow rate is above a certain threshold, such as for example 300 ml/hr, and the mean value of the derivative of the flow rate is above another threshold value, such as 50 (ml/hr)/sec, this flow rate and flow rate derivative indicate a start of a bolus dose. The threshold values are selected based upon the finding that typical bolus dose deliveries have a flow rate between about 50 ml/hr to about 6000 ml/hr, while a human injecting a bolus dose is typically incapable of delivering the injection at a rate less than about 50 ml/hr, on a per second basis.

The outputs of the differential pressure sensor assembly 100 may also be used to monitor both the delivery of medication via a single bolus dose, and via an infusion pump. Such an algorithm would indicate that a flow rate below a threshold level, such as for example 50 ml/hr, is not from a bolus dose. Similarly, infusion pump cycles provide a consistent sinusoidal pattern of deliveries with every pumping cycle. Utilizing an approach that analyzes the output of the sensor assembly 100 in a frequency domain, such as through a Fourier transform, pump infusion cycles appear at a much higher frequency than flow rates introduced through a single bolus dose. A low pass filter with a cutoff frequency separating the frequency band due to an infusion pump action, versus manual delivery via a single bolus dose, can segregate the flow rate signal due to each source. Alternatively, an inverse Fourier transform of the frequencies in the band below the frequencies affected by the pump action can recover a time domain flow rate signal from the differential pressure based sensor assembly 100 to quantify the amount of flow from a single bolus dose. Such an algorithm to isolate flow due to a pump source from flow due to manual injection could also be utilized to verify an infusion pump flow rate. Similarly, pressure pulsations occurring as a result of arterial pulsations when the sensor is in direct fluidic connection with an arterial vessel can be detected and mathematically compensated for using frequency domain low pass filtering below a cutoff frequency, since manual injections are usually lower frequency than arterial pulsations. Alternatively, linear weighted averaging of pressure values measured at the sensor is a form of filtering or smoothing that can be applied on the signal to reduce the effect of pulsations. Typical infusion pumps do not measure flow volume, but rather estimate flow volume based upon pump fluidic displacement. Thus, a differential pressure based flow sensor assembly 100 may verify infusion pump function, or be used in a closed feedback loop to control pump flow rate.

Yet another algorithm contemplated allows the differential pressure based sensor assembly 100 to be used to detect air pockets within fluids flowing through the sensor assembly 100. An air pocket typically is much less dense than a fluid passing through the sensor assembly 100. Thus, an air pocket or bubble within a fluid medium generates an abrupt change in pressure value, followed by a return to expected levels. The start and end of the abrupt change in pressure values is detected by monitoring the first derivative and the second derivative of the output of the upstream pressure sensor 120 and the downstream pressure sensor 122. An abrupt change in pressure would first be noticed on the upstream pressure sensor 120, followed by an abrupt change in pressure on the downstream pressure sensor 122. These pressure changes would be followed by an abrupt resumption back to pressure levels prior to air pocket reception, once the air pocket is passed. The duration of the deviation from typical pressures is indicative of the size of the air pocket.

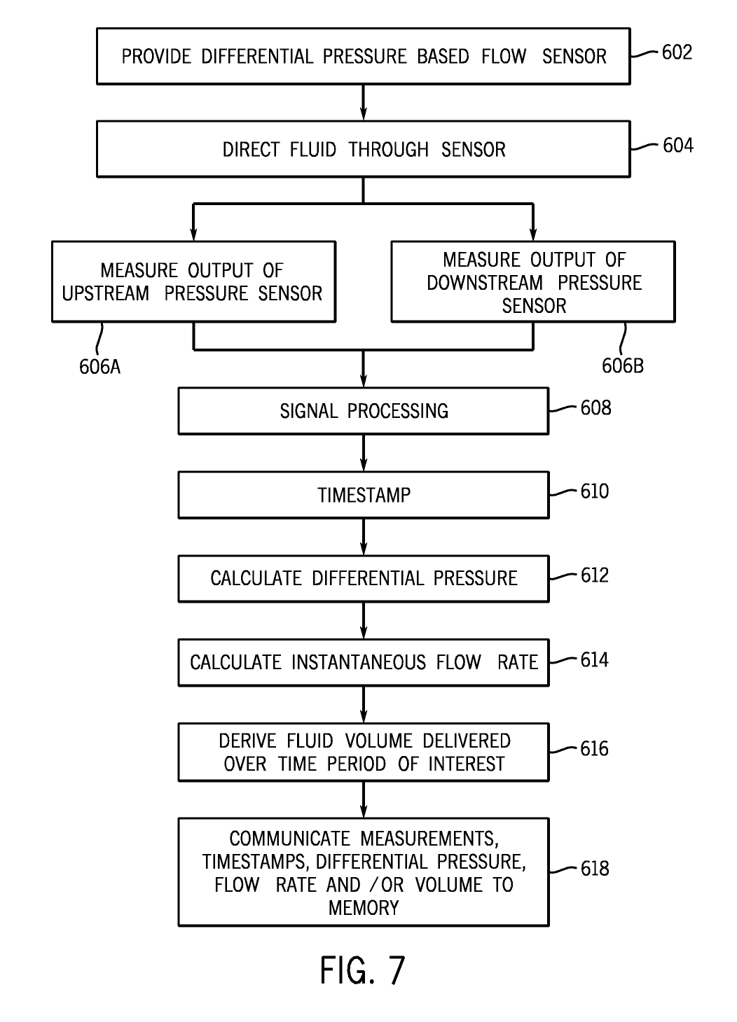

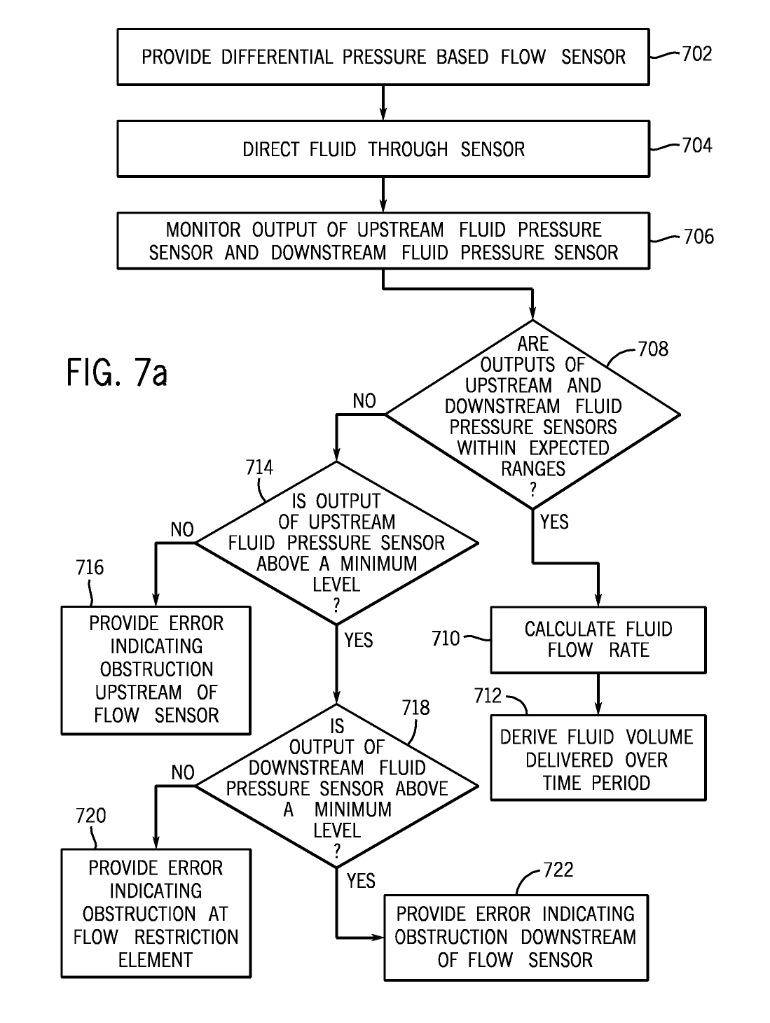

FIG. 7 shows a basic process of utilizing a differential pressure based sensor assembly 100 to determine the instantaneous flow rate and/or volume of a fluid flow delivered through a bolus or other delivery. The process provides a differential pressure based flow sensor assembly 100 in step 602. Fluid flows through the sensor assembly in step 604. The output of the upstream pressure sensor 120 is measured in step 606A, and the output of the downstream pressure sensor 122 is measured in step 606B. The signals from the sensors 120, 122 can be filtered, amplified, or otherwise processed (for example as described above) in step 608. A timestamp is associated with the measurements in step 610. A differential pressure is calculated based upon the observed measurements in step 612. The instantaneous fluid flow rate is calculated in step 614. The flow rate is integrated over time to derive the volume deliver during the time period of interest in step 616. In step 618, the sensor signals or measurements, timestamp information, differential pressure, flow rate and/or volume delivered are communicated to a memory, which can be located in the sensor assembly 100, in the infusion pump 12, or another computer.

Turning now to FIG. 7a, a process of utilizing a differential pressure based sensor assembly to deliver a fluid is depicted, including monitoring for possible occlusions within the delivery system. The process provides a differential pressure based flow sensor in step 702. Fluid flows through the sensor in step 704 and the output of both the upstream fluid pressure sensor and the downstream fluid pressure sensor are monitored in step 706. The process determines whether the outputs of both the upstream fluid pressure sensor and the downstream fluid pressure sensor are within expected ranges in step 708. If so, the process calculates the fluid flow rate, utilizing the algorithm previously described, in step 710. Once the flow rate has been determined, the process derives the volume that has passed through the sensor assembly 100 over a given period of time in step 712. As described above with respect to FIG. 7, the sensor signals or measurements, timestamp information, differential pressure, flow rate and/or volume delivered are communicated to a memory, which can be located in the sensor assembly 100, in the infusion pump 12, or another processor.

If the outputs of the upstream and downstream fluid pressure sensors do not fall within expected ranges, the process determines if the output of the upstream fluid pressure sensor is above a minimum level in step 714. If the pressure is not above a preset minimum level, an error signal is generated in step 716, indicating that a possible obstruction exists upstream of the differential pressure based flow sensor assembly 100. However, if the output of the upstream fluid pressure sensor is above a minimum level, the process in step 718 determines if the output level of the downstream fluid pressure sensor is above a preset minimum level. If the output of the downstream fluid pressure sensor is not above a preset minimum level, an error signal is generated in step 720 that indicates an obstruction may be present at the flow restricting element 112 or upstream thereof. However, if the downstream fluid pressure sensor detects a pressure above the preset minimum level, an error signal is generated in step 722 indicating that an obstruction may be present downstream of the differential pressure based flow sensor assembly 100.

Thus, utilizing the process illustrated in FIG. 7a, the flow rate of a fluid as well as the volume of the fluid delivered through a differential pressure based flow sensor assembly may be calculated, and an error message may be provided when an occlusion occurs.

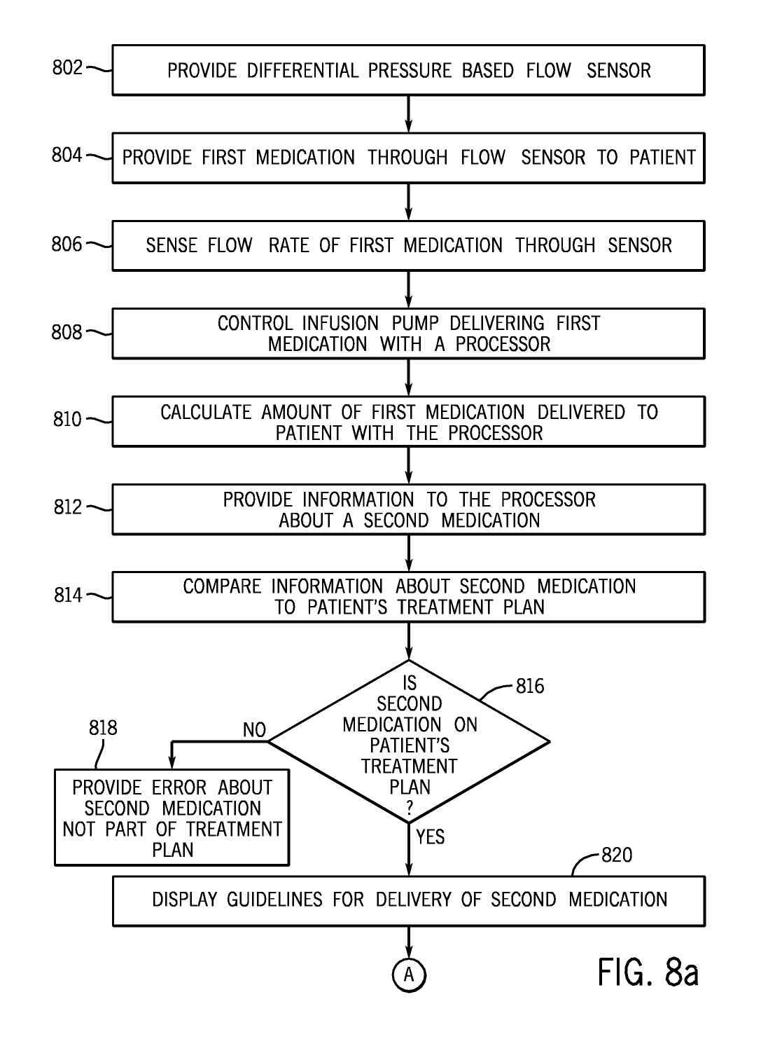

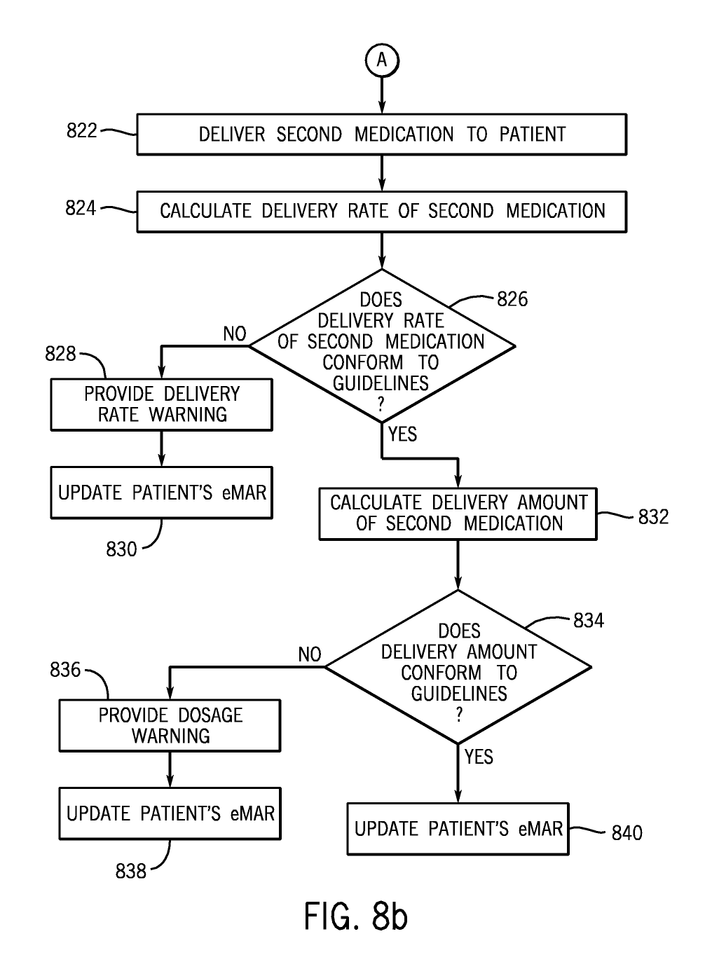

As shown in FIGS. 8a-8b, a method of delivering medication to a patient utilizing a medication delivery system having an infusion pump is depicted in block diagram form. The process provides a differential pressure based flow sensor assembly in step 802, such as sensor assembly 100 previously described herein. A first medication is provided through the flow sensor assembly to the patient 10 in step 804. The flow through the sensor assembly is sensed in step 806. In step 808, the process controls an infusion pump delivering the first medication via a processor. The amount or volume of the first medication delivered to the patient is calculated in step 810 using the processor and signals received from the differential pressure based flow sensor assembly 100. Information about a second medication to be delivered to the patient is provided to the processor in step 812. The information provided about the second medication is compared to information within the patent's treatment plan in step 814. Information about the patient's treatment plan can be stored in a memory of the pump 12, a memory of the reusable portion 104, or another memory or computer in communication with the pump 12 and/or flow sensor assembly 100. The process determines in step 816 whether the second medication is on the patient's specific treatment plan, such as by checking whether the patient has a medical order or prescription for the second medication. If the second medication is not found on the patient's treatment plan, an error message is provided in step 818 indicating that the second medication is not found on the patient's treatment plan, and the caregiver should check with a physician or other caregiver to determine if it is appropriate to provide the second medication to the patient. If the second medication is found on the patient's treatment plan, guidelines for delivering the second medication are generated or displayed in step 820. The guidelines can include but are not limited to a target delivery rate with upper and/or lower limits, a total volume or amount to be delivered during the bolus, and a time period over which to deliver the IV push or bolus.

Continuing now to FIG. 8b, the second medication is delivered to the patient in step 822. The process calculates the delivery rate of the second medication using the differential pressure based flow rate sensor assembly 100 in step 824. As described with respect to FIG. 7 above, the delivery flow rate calculations can be stored in memory. A comparison is performed in step 826 to determine if the delivery rate of the second medication conforms to the delivery guidelines. If the delivery rate does not conform to the delivery guidelines, a delivery rate warning is provided to the caregiver in step 828. If the delivery rate warning is provided, the patient's electronic medication administration record (eMAR) is updated in step 830 to show that the second medication was delivered at a rate inconsistent with the delivery guidelines or protocols. The amount of the second medication delivered to the patient can also be calculated in step 832. The process in step 834 compares the amount of the second medication delivered to the amount of the second medication the patient was scheduled to receive. If the amount of the second medication the patient received does not conform to the patient's treatment plan, a dosage warning is provided to the caregiver at step 836. This warning can indicate that the patient was provided an underdose of the second medication, or that the patient was provided with an overdose of the second medication. The patient's electronic medication administration record (eMAR) is updated in step 838 to include the amount of the second medication that was provided to the patient, as well as information to indicate that the dosage of the second medication did not conform to the patient's treatment plan. If the amount of the second medication delivered to the patient conforms to the patient specific guidelines, the patient's electronic medication administration record (eMAR) is updated in step 840 to indicate that a proper dosage of the second medication was delivered to the patient. It is contemplated that every update to the patient's electronic medication administration record (eMAR) will note the time a medication was delivered to the patient, as well as the caregiver responsible for delivering that medication to the patient.

According to a further embodiment, a disposable infusion tubing set is provided that has a disposable portion of a differential pressure based flow sensor assembly. The tubing set would include at least a first tube adapted to connect to a first medication reservoir, and a connection site to allow a second medication to be introduced into the first tube of the tubing set upstream of the disposable portion of the differential pressure based flow sensor assembly. The disposable infusion tubing set further has a second tube adapted to connect to a patient access device. The second tube is adapted to be positioned downstream of the disposable portion of the differential pressure based flow sensor assembly. As discussed above, the disposable portion of the differential pressure based flow sensor assembly can be disposed in other locations within the disposable infusion tubing set, depending on the line pressure conditions, delivery flow rates, or fluid volume delivery amounts of interest.

According to yet another embodiment, a differential pressure based flow rate sensor assembly can serve as or be replaced by a pressure based event detection sensor. A pressure based event detection sensor allows an event, such as a bolus, to be detected noting a spike in pressure. Such an event detection sensor would not allow the computation of the volume of medication delivered, but will place a notation onto a patient's record that some medication was delivered at a specific time. Thus, a record will exist confirming that a patient was provided with medication.

According to yet a further embodiment, a differential pressure based flow sensor assembly may be powered by an inductive power source. Such an embodiment would contain many of the same features as the differential pressure based flow sensor assembly 100 described herein. Similarly, it is contemplated that a wireless differential pressure based flow sensor assembly may transmit information regarding a pressure at an upstream pressure sensor and information regarding a downstream pressure sensor to other components within a system. Finally, it is contemplated that the portion 104 of the differential pressure based flow sensor assembly 100 could be produced using MEMS, integrated circuits or other technology in a miniaturized and low cost manner, such that the portion 104 might be considered disposable as well.

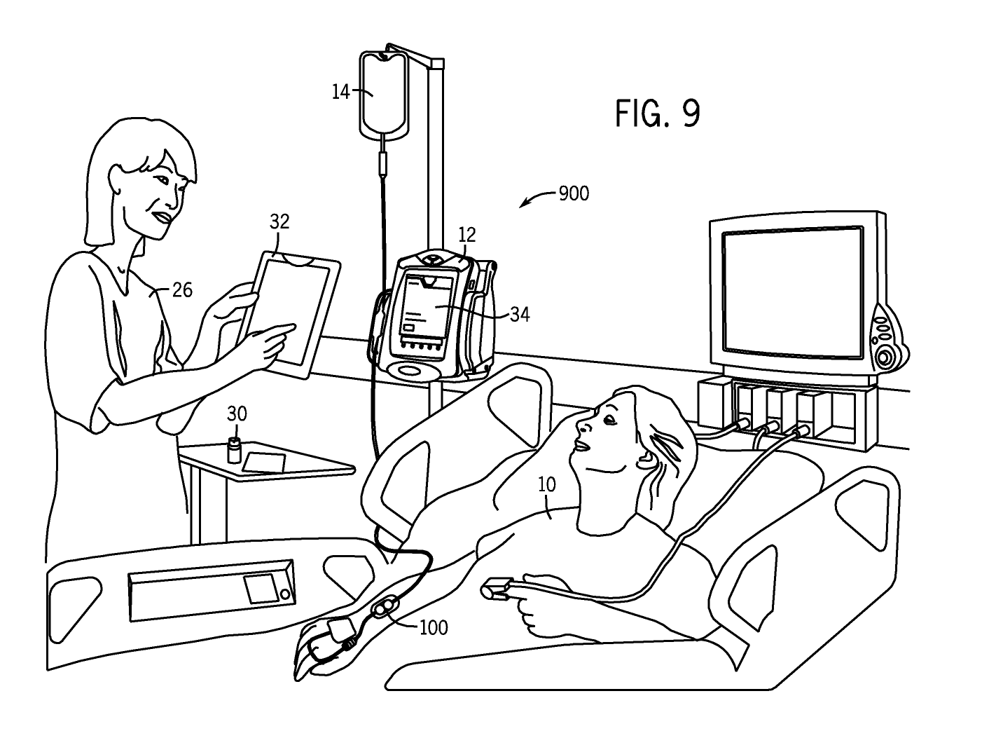

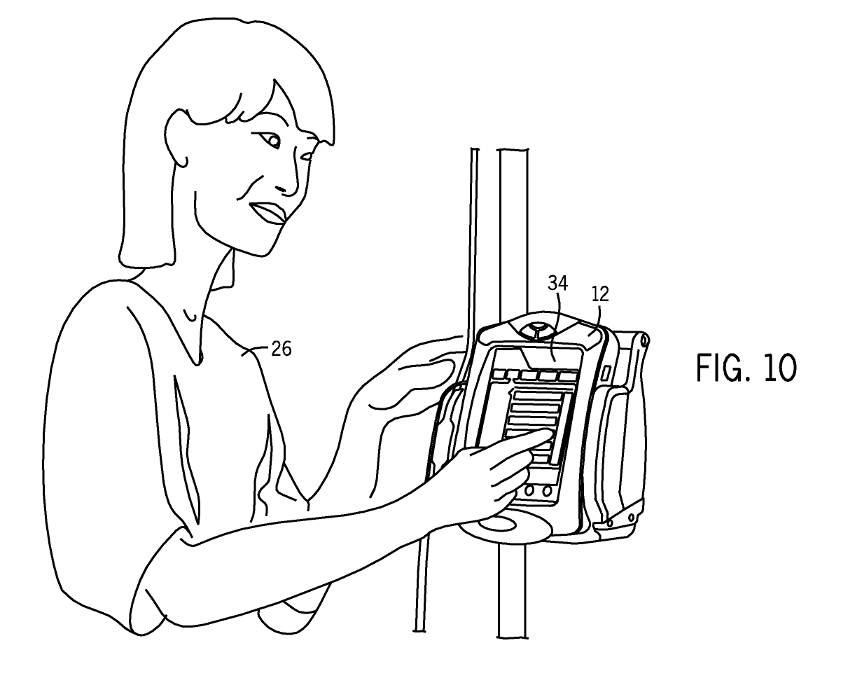

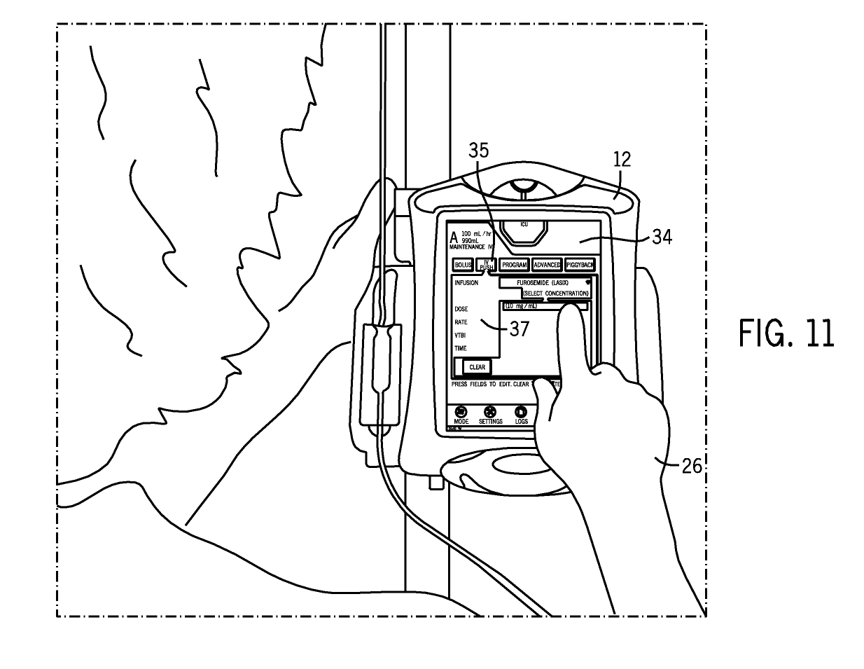

Turning now to FIGS. 9-14, a method of delivering a medication to a patient is pictorially depicted using the medication delivery system 1. As shown in FIG. 9, the caregiver 26 approaches the patient 10 with a medication 30 and a medical administration record 32 (“MAR”) for the patient 10. The caregiver 26 next confirms that the identity of the patient 10 and the name on the MAR 32 match. The caregiver 26 may view an interface 34 on the infusion pump 12 to assist in verifying the identity of the patient 10, in addition to talking with the patient 10, or viewing or scanning other identifying information found on the patient 10, such as a hospital identification bracelet or other similar identifying indicia.

Turning next to FIGS. 10 and 11, once the identity of the patient 10 is confirmed, the caregiver 26 utilizes the interface 34, which preferably is a touch-screen display, on the pump 12 to select the medication 30 that the caregiver 26 approached the patient 10 to deliver. The interface 34 has an “IV-Push” button 35 that when activated by the user provides an IV-Push therapy option or mode and parameter input/display area 37. The interface 34 may display a plurality of medications and concentrations that have been prescribed for the patient 10, thus the caregiver will properly select the medication 30 from the choices present on the interface 34. Additionally, once the medication 30 is selected, the system 1 may be adapted to display a message indicating if the medication 30 may cause an adverse reaction with another medication the patient 10 is receiving, or if the patient 10 may suffer an allergic reaction to the medication 30.

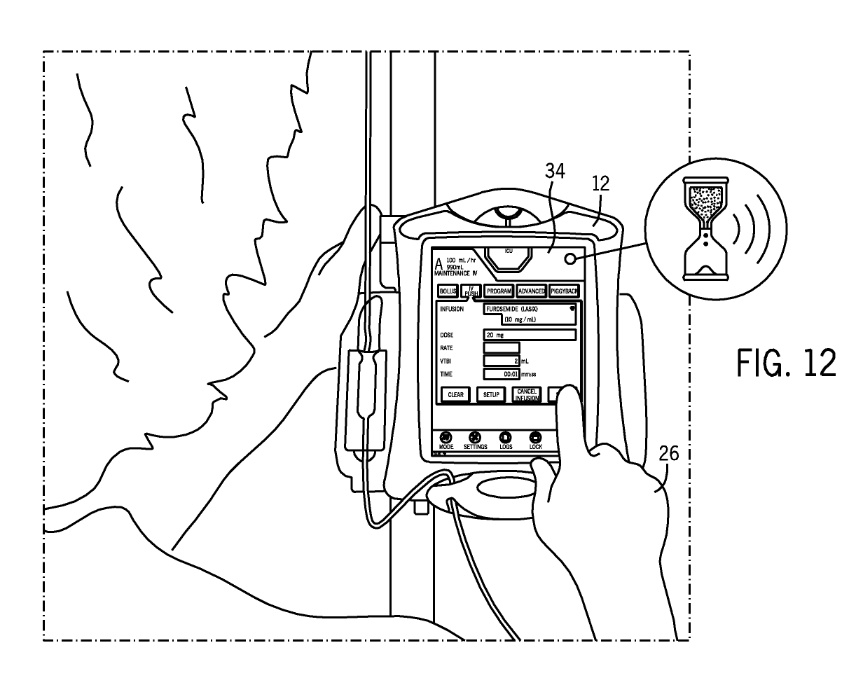

As depicted in FIG. 12, once the medication 30 is selected on the interface 34, the pump 12 displays a confirmation screen on the interface 34. The confirmation screen allows the caregiver 26 to enter the amount of medication 30 that will be delivered to the patient, and the system 1 calculates a recommended period of time for the caregiver 26 to deliver the medication 30, and displays this information to the caregiver 26 on the interface 34. Certain medications require delivery to a patient 10 in a time sensitive manner, while other medications are not as dependent on the delivery rate.

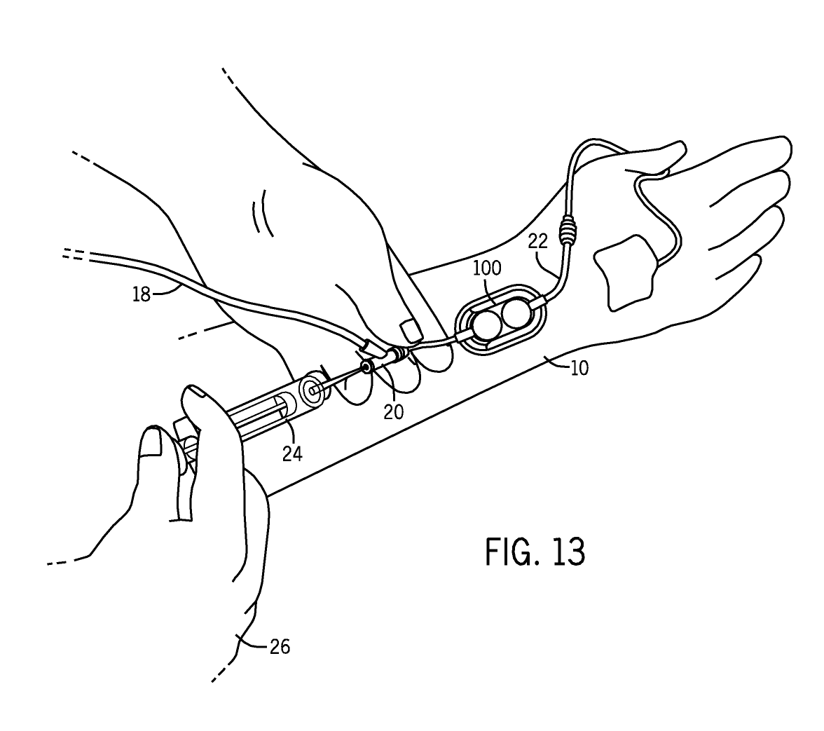

The caregiver next places the medication 30 into a syringe 24 for injection into the patient 10, as depicted in FIG. 13. The caregiver 26 connects the syringe 24 to the second fluid line 18 via the connection 20. The caregiver 26 then delivers the mediation within the syringe 24 to the patient 10 through the connection 20. The medication passes through the differential pressure based fluid flow sensor 100 and the third fluid line 22 to the patient 10. The differential pressure based fluid sensor assembly 100 monitors the flow rate of the medication through the sensor assembly 100. By monitoring the flow rate through the sensor assembly 100, the volume of medication delivered to the patient 10 may be calculated. Monitoring the flow rate allows the interface 34 to instruct the caregiver 26 to either increase the speed at which the medication is being delivered, or slow the speed at which medication is being delivered.

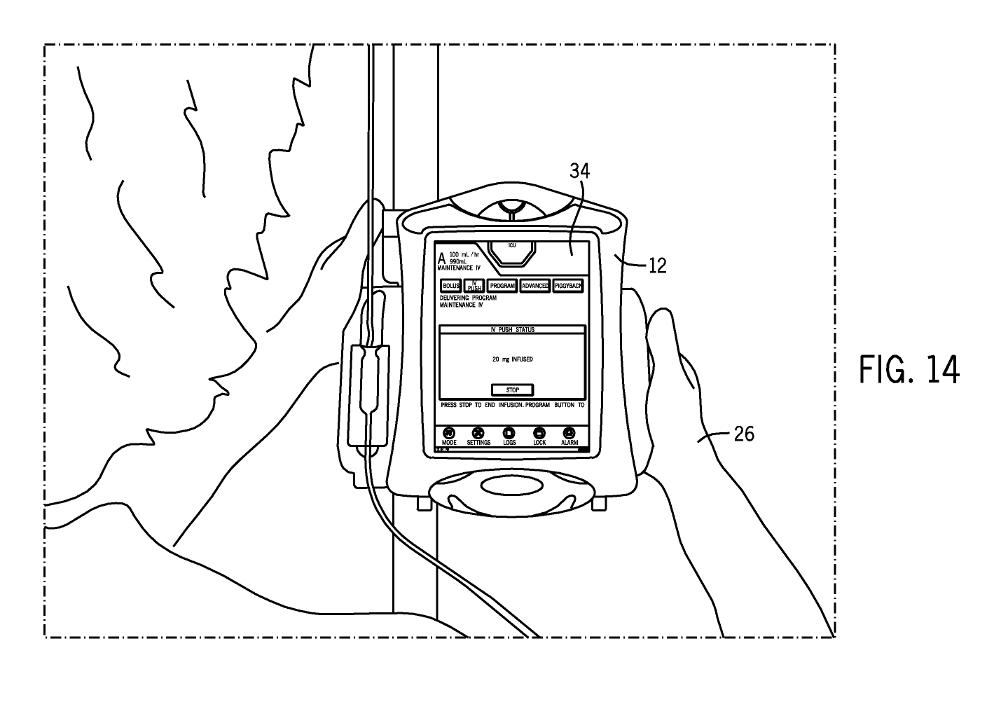

Once the medication 30 has been infused into the patient, the caregiver 26 may again interact with the interface 34 of the pump 12. Turning to FIG. 14, the interface 34 displays the amount of medication that the caregiver 26 has delivered to the patient based on data obtained from the flow sensor 100. The hospital may configure the pump 12 via a customizable, electronically downloadable drug library that defines which drugs can be delivered by IV push, the best practices IV push rate for each drug that is allowed to delivered by IV push, the desired customized rounding and/or truncation rules, as well as units and number of digits to display or communicate. The system 1 may then update the patient's 10 eMAR to reflect the delivery of the medication 30.

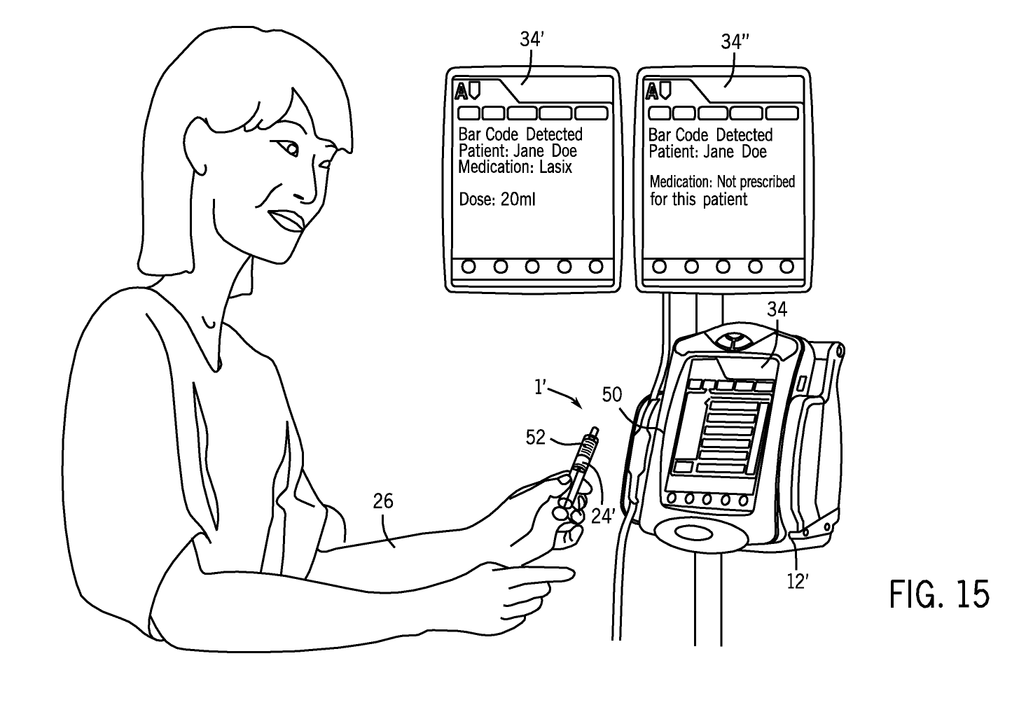

As shown in FIG. 15, it is contemplated according to an alternate embodiment that a system 1′ may utilize a pump 12′ that features a bar code reader 50. Medication 30 may be provided in a syringe 24′ that features a bar code 52 on the exterior of the syringe 24′. The syringe 24′ is positioned so that the bar code 52 may be read by the bar code reader 50. As shown on interface 34′ the identity of medication within the syringe 26′ and optionally an identifier of the patient can be displayed to the user 26 once the bar code 52 is detected. As shown on interface 34″ a warning message is provided to the caregiver 26 indicating that the medication within the syringe 24′ has not been prescribed for the patient. The system 1′ reviews a patient's eMAR to determine if the medication has been prescribed for the patient.

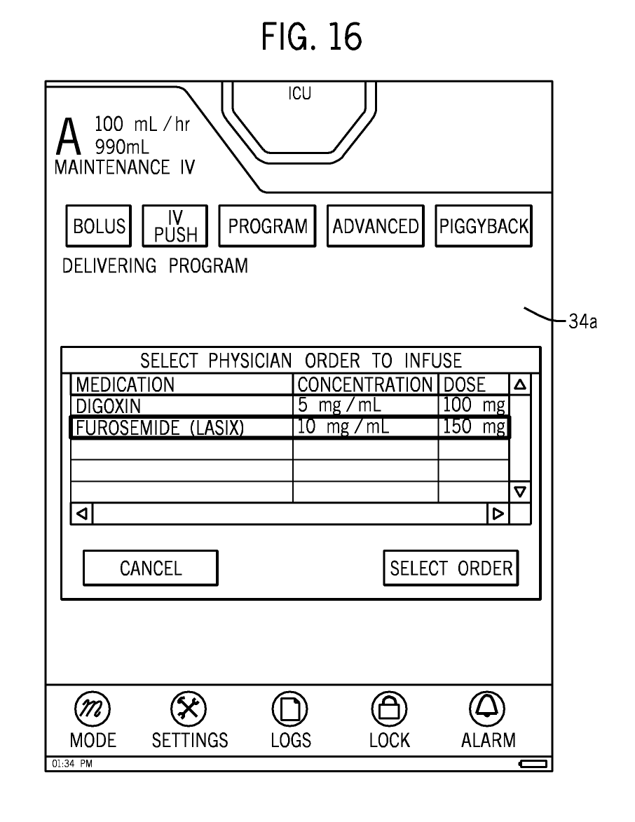

Turning now to FIGS. 16-28, specific displays of information that may be shown on the interface 34 are depicted. FIG. 16 shows an interface screen 34a displaying two drugs that have been prescribed for a patient. The caregiver may select one of the orders and the associated medication as a medication to be delivered to the patient.

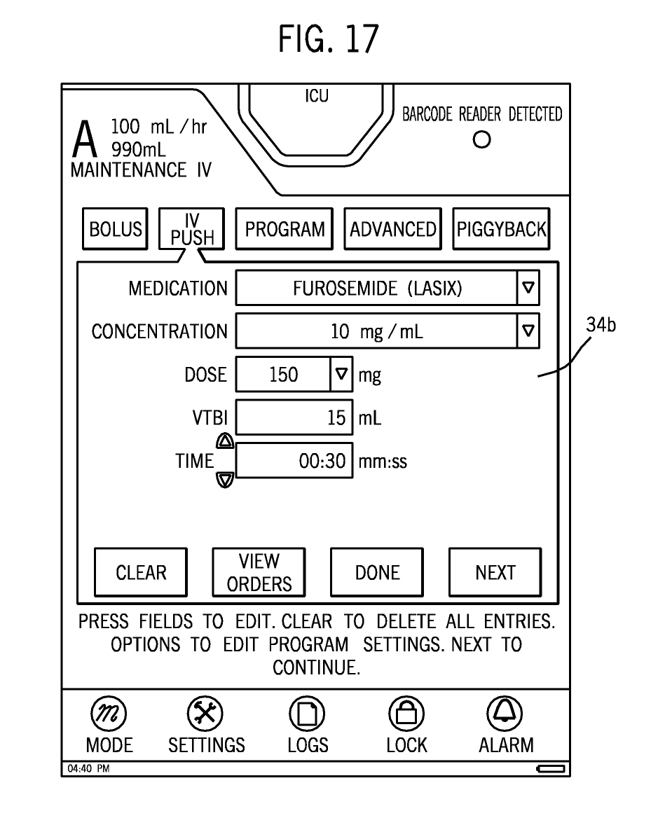

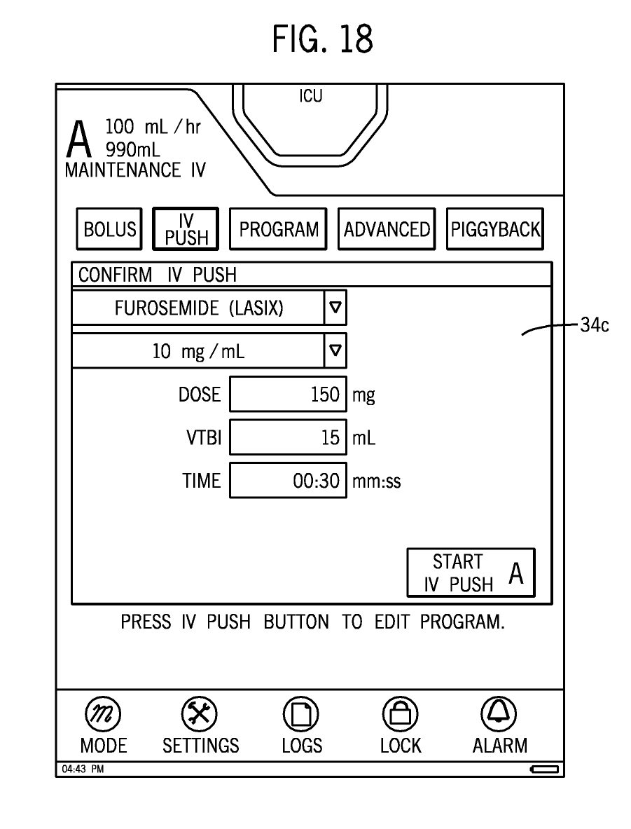

FIG. 17 shows an interface screen 34b after the medication has been selected on the interface screen 34a. The interface screen 34b allows the caregiver to select the length of time over which the medication will be delivered, and indicates to the caregiver the volume of medication that is required to provide the prescribed dose. Alternatively, the delivery parameters (rate, time, volume or dosage, etc.) can be read or scanned from the label of the medication 30 and communicated to the pump 12 from the reader 50 or an information system within the hospital, including but not limited to a bar code point of care system or a pharmacy information system. In the case of manual or semi-manual versus automated programming, after the caregiver selects the amount of time in which to deliver the medication and inputs any missing parameters, an interface screen 34c appears, as shown in FIG. 18, which allows the caregiver to confirm the information previously provided and initiate the infusion by pressing start. Of course, if the parameters are automatically provided, the user can be presented with the confirmation screen without delay.

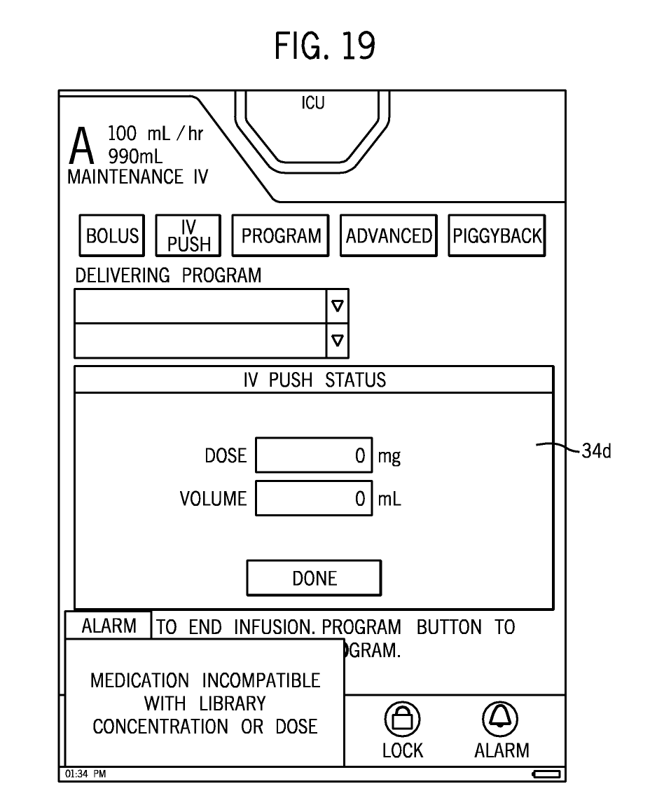

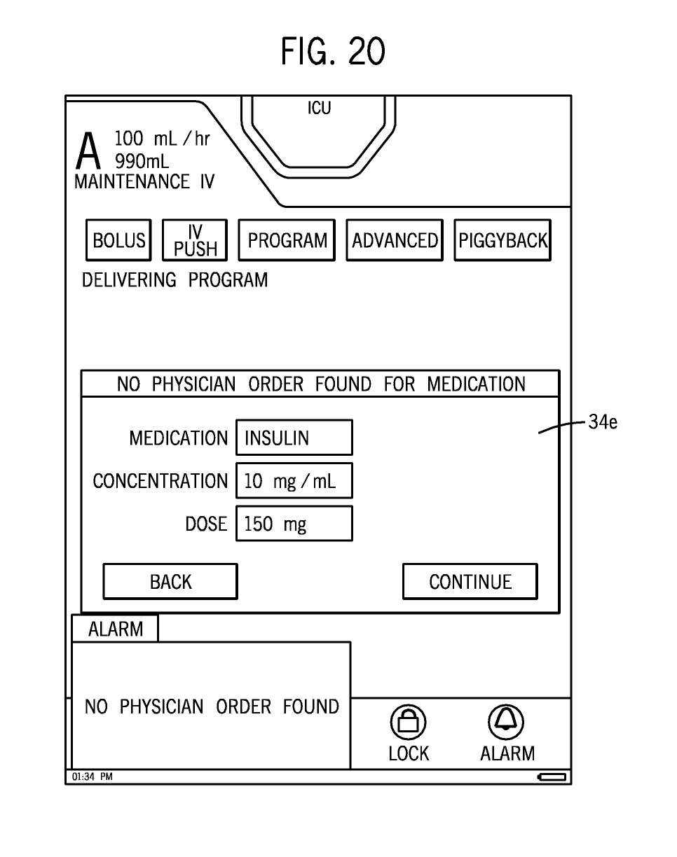

According to one method, once the caregiver presses start, the medication delivery system 1 verifies that the medication dosage is correct, and verifies once again that the medication has been prescribed for the particular patient. FIG. 19 shows an interface screen 34d that contains an alarm message, or warning message, that indicates either the concentration of the medication selected is not found in the system, or the dose of the medication to be delivered is not within the system. For example, if a medication is not available in 10 mg/ml concentration, but that concentration was selected, such an error would be displayed. Similarly, if a dose of 150 mg is not an appropriate dose for a particular patient, such a warning as shown on the interface screen 34d may appear. Similarly, as shown on interface screen 34e of FIG. 20, if a medication selected by a caregiver has not been prescribed for a patient, an alarm message, or warning message, is displayed to the caregiver indicating that an order has not been entered into the eMAR for the patient to receive that particular medication. Thus, these warnings provide a caregiver with an additional check to verify that the patient is supposed to be receiving a particular medication, and that the concentration and dose of the medication are proper, prior to the caregiver delivering a medication to a patient

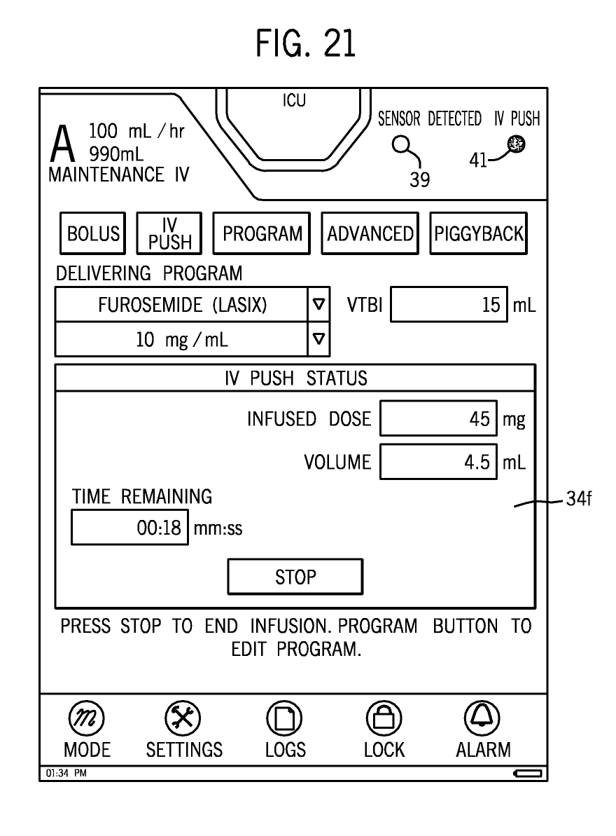

Once the caregiver begins delivering medication to the patient, an interface screen 34f may be displayed, as shown in FIG. 21. The interface screen 34f displays the medication being delivered, the concentration of the medication, and the total volume of the medication the patient should receive (VTBI—volume to be infused). Additionally, the interface screen 34f displays the dose and the volume of the medication that has been delivered since the infusion began, and the total time remaining for the caregiver to complete the infusion of the medication based on the delivery guidelines. The interface screen 34f may also alert the caregiver to the fact that a flow sensor has been detected by the system as evidenced by indicator 39, and that the flow sensor is currently observing fluid flow as evidenced by indicator 41.

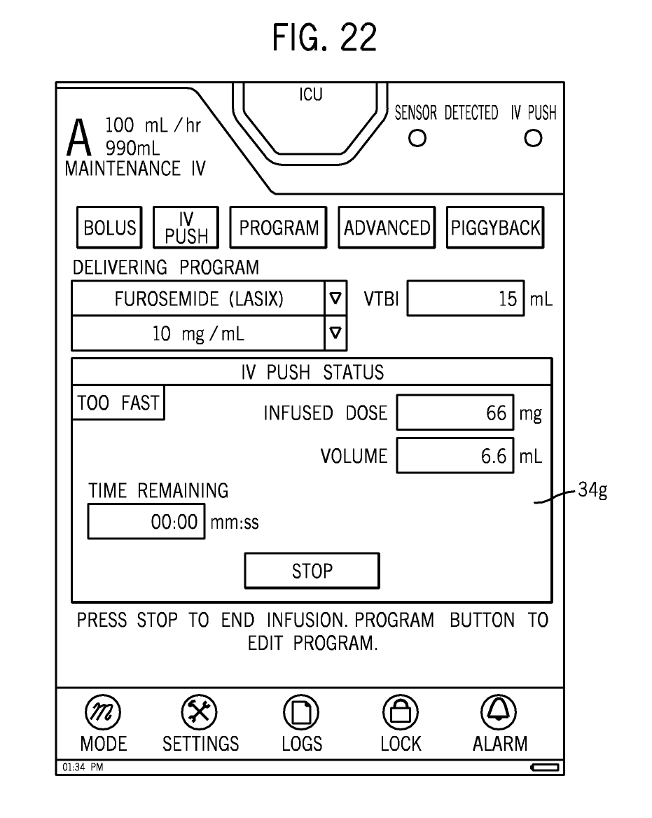

While the caregiver is delivering the medication to the patient, an interface screen 34g, depicted in FIG. 22, can be generated to provide a warning to the caregiver that medication is being delivered too quickly. The data from the flow sensor allows the system monitor the flow rate in real-time, or nearly real-time, such that for medication with a specified delivery rate, the caregiver may be informed that the observed rate is too fast. Similarly, it is contemplated that an interface screen could be provided to alert the user that the medication delivery rate is not fast enough, prompting or causing the caregiver to increase the medication delivery rate. It is still further contemplated that a graphical display, including but not limited to a bar graph or tachometer-style display, could be provided that provides the caregiver with graphical flow rate boundaries that the medication flow rate should be kept within. Colors such as green for acceptable, yellow for warning or near-unacceptable, and red for alarming or unacceptable push or flow rates can be utilized. Providing the caregiver with visually guidelines may help insure proper medication delivery rates. Similar tools can be utilized for volume delivered.

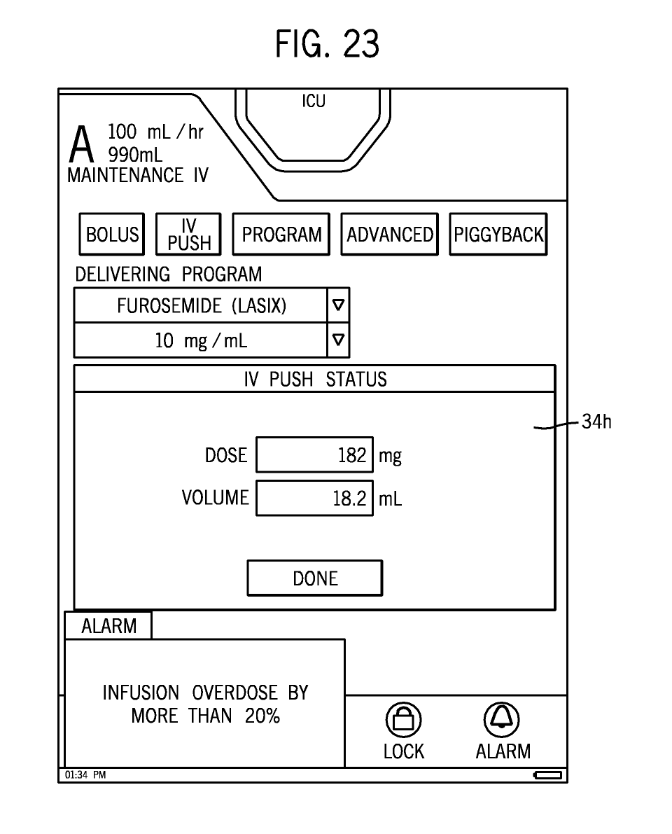

Turning now to FIG. 23, an interface screen 34h is depicted having an alarm, or warning, indicating that an overdose of medication has been provided to the patient. The system may be set by a hospital, or other facility, to provide an overdose alarm if the dose provided to the patient is a certain percentage above the prescribed dose. The hospital can customize or configure the pump via a drug library. The alarm may be configured to vary based on the medication delivered to the patient. The percent overdose required for the interface screen 34h providing an overdose warning may be less than twenty percent (20%) for certain dose critical medications, and more than twenty percent (20%) for other, less dose critical medications.

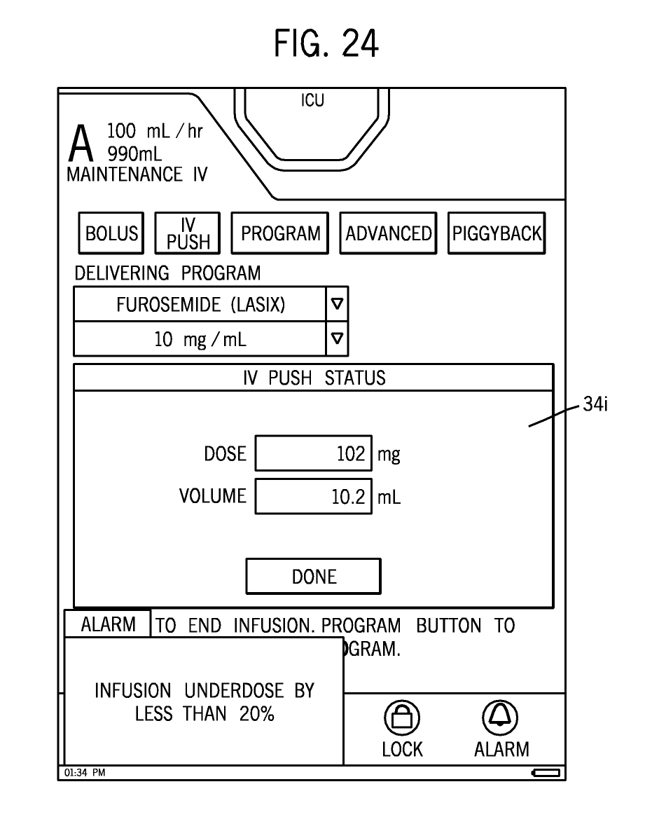

Similarly, FIG. 24 displays an interface screen 34i having an alarm or warning indicating that an underdose of medication was provided to the patient. As with the overdose alarm, the underdose alarm may be configured by the hospital, and may vary based on the medication.

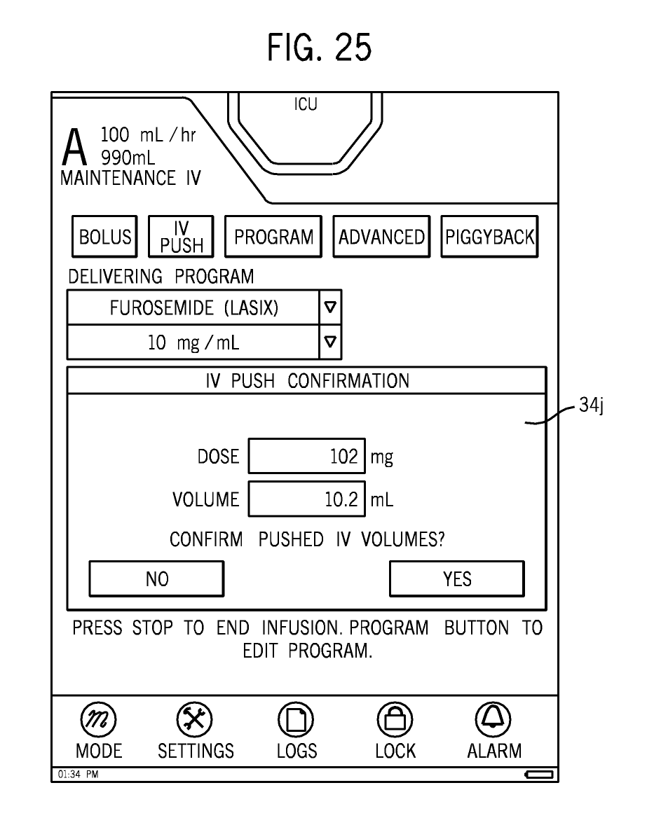

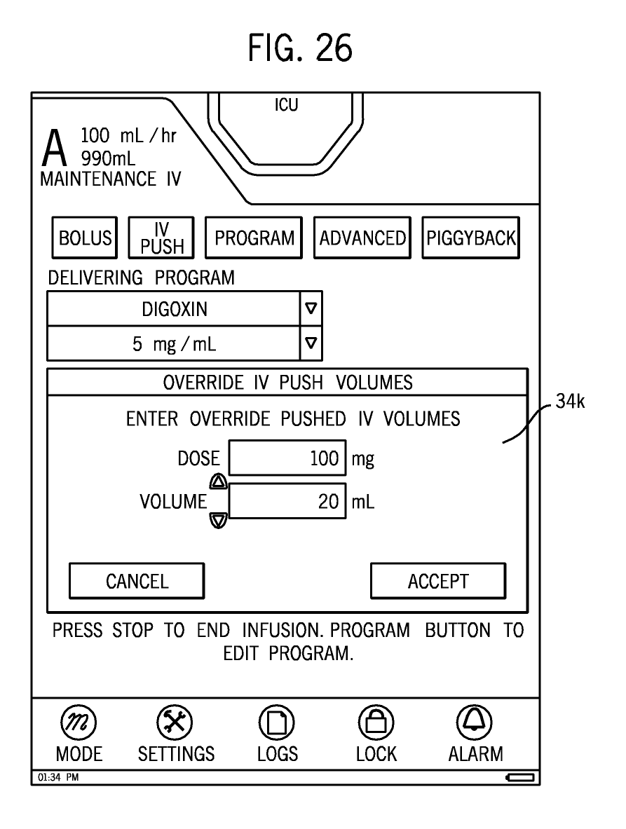

Once the medication delivery is complete, an interface screen 34j (FIG. 25) is provided to allow the caregiver to confirm the volume of medication that has been delivered to the patient. Once the caregiver has confirmed the volume of medication detected by the system, the caregiver may manually edit the information. As shown on interface screen 34k in FIG. 26, the caregiver may edit or override the volume information to reflect the prescribed dose, or simply make the volume delivered a whole number. Settings within the system may limit the number of caregivers that may use the override screen, such that only a physician or a nursing supervisor may edit the information. Additionally, even if the information is edited on the override interface screen 34k, the patient's eMAR is updated with the information provided by, or calculated from, the flow sensor of the system. In this manner, exact details of the medication delivery will always be stored within the eMAR.

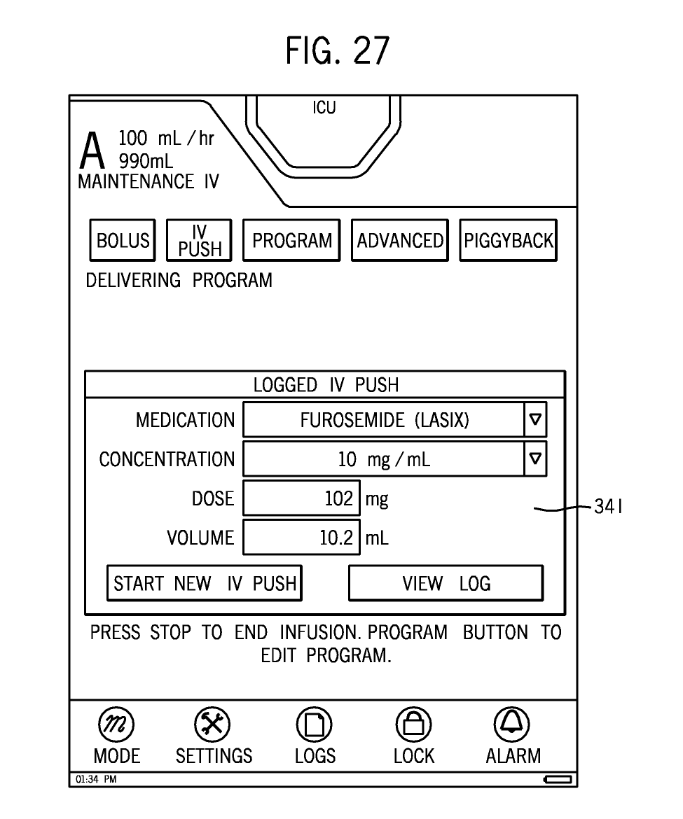

Turning next to FIG. 27, another interface screen 34l is depicted showing the volume and dose of medication that has been delivered to the patient once the caregiver has confirmed the volume of medication provided to the patient on the interface screen 34h. This information is entered in the patient's eMAR, and may be observed by the caregiver by selecting the View Log option on the interface screen 34l.

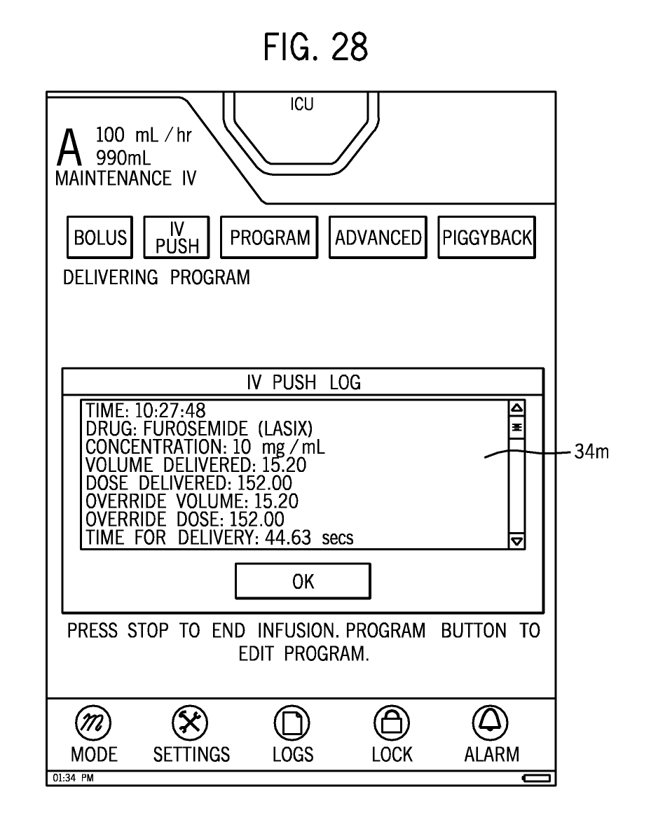

Finally, if the caregiver selects the View Log option from interface screen 34l, a medication delivery log interface screen 34m will be displayed as shown in FIG. 28. The medication delivery log interface screen 34m provides pertinent information regarding the delivery of medication to the patient, including the time of the delivery, the medication delivered, the concentration of the medication, the volume of medication delivered, the dose of the medication delivered, whether an override of the sensor generated information was entered, and the duration of the medication delivery. This information may be sent to the patient's eMAR, such that a complete record of every medication the patient receives is properly maintained.

While the foregoing has described what is considered to be the best mode and/or other examples, it is understood that various modifications may be made and that the subject matter disclosed herein may be implemented in various forms and examples, and that they may be applied in numerous other applications, combinations and environments, only some of which have been described herein. Those of ordinary skill in that art will recognize that the disclosed aspects may be altered or amended without departing from the true scope of the subject matter. Therefore, the subject matter is not limited to the specific details, exhibits and illustrated examples in this description. It is intended to protect any and all modifications and variations that fall within the true scope of the advantageous concepts disclosed herein.

We claim:

1. A method of delivering medication to a patient utilizing a medication delivery system comprising:
supplying at least a first medication to be delivered to the patient;
verifying an identity of the patient;
selecting the first medication on a user interface for delivery to the patient;
entering the volume of the medication to be delivered to the patient on the user interface;
injecting the first medication into the patient through a flow sensor assembly;
monitoring a flow rate and a volume of the first medication delivered to the patient during the injection using the flow sensor assembly;
providing a visual display regarding the act of injecting on the user interface; and
updating the patient's electronic medical administration record to capture the injecting of the first medication.

2. The method of claim 1, wherein the monitoring of the flow rate and a volume of the first medication delivered to the patient uses a differential pressure based flow sensor assembly.

3. The method of claim 1, wherein the act of selecting on the user interface utilizes a touch-screen display.

4. The method of claim 1 further comprising displaying on the user interface a recommended delivery duration for the first medication.

5. The method of claim 1 further comprising warning on the user interface of any potential adverse interactions between the first medication and any other medications the patient is receiving.

6. The method of claim 1 further comprising warning on the user interface of any potential patient allergy to the first medication.

7. The method of claim 1 further comprising instructing that the injecting of the first medication be slowed down based on the monitoring of the flow rate by the flow sensor assembly.

8. The method of claim 1 further comprising determining the conclusion of the injecting of the first medication; and
warning if the volume of the first medication delivered to the patient does not match the volume of medication entered for the patient to receive.

9. A method of delivering medication to a patient utilizing a medication delivery system comprising:
supplying at least a first medication to be delivered to the patient;
verifying an identity of the patient;
scanning a bar code on the first medication;
displaying the identity of the first medication based on the scanning of the bar code;
confirming that the first medication is prescribed for the patient;
entering the volume of the medication to be delivered to the patient;
injecting the first medication into the patient through a flow sensor assembly;
monitoring a flow rate and a volume of the first medication delivered to the patient during the injection using the flow sensor assembly;
providing a visual display regarding the act of injecting; and
updating the patient's electronic medical administration record to capture the injecting of the first medication.

10. The method of claim 9, wherein the monitoring of the flow rate and a volume of the first medication delivered to the patient uses a differential pressure based flow sensor assembly.

11. The method of claim 9 further comprising displaying a recommended delivery duration for the first medication.

12. The method of claim 9 further comprising warning of any potential adverse interactions between the first medication and any other medications the patient is receiving.

13. The method of claim 9 further comprising warning of any potential patient allergy to the first medication.

14. The method of claim 9 further comprising instructing that the injecting of the first medication be slowed down based on the monitoring of the flow rate by the flow sensor assembly.

15. The method of claim 9 further comprising determining the conclusion of the injecting of the first medication; and
warning if the volume of the first medication delivered to the patient does not match the volume of medication entered for the patient to receive.

16. The method of claim 9 further comprising overriding data generated during the monitoring of the flow rate and the volume of the first medication delivered to the patient to modify the volume and dose of the first medication delivered; and
updating the patient's electronic medical administration record to capture the overriding such that the patient's electronic medical administration record captures both the data generated during the monitoring of the injecting of the first medication and the overriding data entered.

17. The method of claim 16 further comprising displaying at least one of the flow rate, the volume, and a differential pressure from the flow sensor assembly on a user interface of the medication delivery system.

18. A method of delivering medication to a patient utilizing a medication delivery system comprising:
receiving information regarding an identity of a first medication;
displaying the identity of the first medication;
verifying that the first medication has been prescribed for a patient;
monitoring a flow rate and a volume of the first medication delivered to the patient during the injection from a flow sensor assembly;
providing a visual display regarding information obtained from the monitoring of the flow rate and the volume of the first medication delivered to the patient;
updating the patient's electronic medical administration record to capture information regarding the delivery of the first medication.
